(19) United States
(12) Patent Application Publication
(10) Pub. No.: US 2023/0062769 A1
(43) Pub. Date: Mar. 2, 2023

(54) SYSTEM AND METHOD FOR ENDOSCOPE PROPULSION AND LUMEN EVACUATION

(71) Applicant: OTTEK LTD., Nazareth (IL)

(72) Inventor: Yoav LICHTENSTEIN, Hod Hasharon (IL)

(73) Assignee: OTTEK LTD., Nazareth (IL)

(21) Appl. No.: 17/796,939

(22) PCT Filed: Feb. 3, 2021
(86) PCT No.: PCT/IL2021/050127

§ 371 Date: Aug. 2, 2022

(30) Foreign Application Priority Data
Feb. 3, 2020 (IL) 272441

(51) Int. Cl. A61B 1/00

(52) U.S. Cl. CPC A61B 1/0016; A61B 1/00135; A61B 1/00149; A61B 1/00128; A61B 1/31

(57) ABSTRACT

System and method for advancing and retracting a tubular medical instrument within a body lumen and evacuating lumen material therefrom. A shaft of the medical instrument is inserted through a central bore of a probe, an evacuation adaptor, and a swivel fastener unit, respectively mounted onto a distal carriage, a central carriage, and a proximal carriage of a translation mechanism. The probe is directed toward cavity entrance of body lumen using an adjustment mechanism coupled to a translation mechanism beam. A central probe unit of probe is maintained sealingly coupled to cavity entrance by propelling distal carriage along beam. The evacuation adapter is maintained sealingly coupled to the probe by propelling central carriage along beam, concurrent with displacement of a hollow tube of probe relative to central probe unit. Lumen material is evacuated using evacuation adaptor. The medical instrument is advanced and retracted by propelling proximal carriage along beam.

### FIELD OF THE INVENTION

The present invention relates generally to the propulsion of objects within a body lumen, and specifically to methods and devices for propelling tubular medical instruments in a body lumen, such as a colon, and evacuating fluid material therefrom.

### BACKGROUND OF THE INVENTION

The diagnostic and therapeutic advantages conferred by direct examination of a body cavity, and specifically the gastrointestinal tract, with a flexible endoscope, have made this a standard procedure of modern medicine. One of the most common endoscopic procedures is a colonoscopy, which may be performed for a variety of purposes, such as: diagnosis of ailments or diseases; including cancer; determining the source of gastrointestinal bleeding; examining a site affected by inflammatory bowel disease; removing polyps; reducing volvulus and intussusception; and other medical objectives. Endoscopy and colonoscopy generally involve the maneuvering of a flexible endoscope, or similar prevalent endoscopic devices, within a body cavity. The maneuvering, and more particularly the insertion and extraction, of the endoscope requires substantial effort and aptitude by a proficient physician or medical professional performing the procedure (e.g., an endoscopist).

An example of an existing endoscopic apparatus is disclosed in PCT application publication WO2016/075682A1 to Lichtenstein, entitled “Hollow probe with sleeve”, which teaches a probe which enables insertion and accommodation of endoscopic instruments. The probe features a tubular sleeved sheath slidingly disposed inside a central bore of central valve unit having a valve for controlling the introduction and withdrawal of fluid, where the sleeved sheath is covered by a flexible sleeve folded over to cover both the inside and outside of the sheath to sealingly envelop the sheath and contain the fluid together with the valve unit. Another endoscopic apparatus is disclosed in PCT application publication WO2019/077609A1 to Lichtenstein, entitled “Hollow probe with sleeve”, which teaches an endoscope adapter mountable to a central valve unit. The endoscope adapter allows insertion of an endoscope into a sleeve-covered-sheath while sealing the gaps between the endoscope and the sleeve-covered-sheath for retaining body fluids from escaping and enabling the evacuation of the body fluids through an evacuation port.

While colonoscopy is a useful and effective procedure, it is difficult for the medical practitioner to perform because of the need to execute complex maneuvering using his/her right hand (or dominant hand) while controlling the angulation of the endoscope, while using his/her left hand (or weaker hand) for other required actions such as turning an irrigation knob. Besides the accumulated fatigue resulting from the continuous maneuvering of the endoscope, the medical practitioner must also release an endoscopic shaft with the right hand in order to assist the angulation activities executed with the left hand (e.g., for pulling an up-down movable knob and a left-right movable knob). Upon releasing the shaft, the medical practitioner cannot withdraw or push the endoscope, thereby compromising the ability of the endoscope to be effectively utilized for examination and diagnosis (e.g., for identifying polyps in the colon of the patient).

### SUMMARY OF THE INVENTION

In accordance with one aspect of the present invention, there is thus provided a system for advancing and retracting a tubular medical instrument within a body lumen of a patient and evacuating lumen material therefrom. The system includes a probe, an evacuation adapter, a swivel fastener unit, a translation mechanism and an adjustment mechanism. The translation mechanism includes a beam, a distal carriage, a central carriage, and a proximal carriage. The probe includes a hollow tube and a central probe unit through which the hollow tube is moveably inserted, and is configured to hold a shaft of the tubular medical instrument. The evacuation adaptor is disposed proximal to the probe, and is configured to hold the shaft of the tubular medical instrument, and to enable the evacuation of lumen material. The swivel fastener unit is disposed proximal to the probe, and is configured to hold the shaft of the tubular medical instrument. The distal carriage is configured to mount the probe, and includes a distal drive configured to propel the displacement of the distal carriage along the beam. The central carriage is configured to mount the evacuation adaptor, and includes a central drive configured to propel the displacement of the central carriage along the beam. The proximal carriage, is configured to mount the swivel fastener unit, and includes a proximal drive configured to propel the displacement of the proximal carriage along the beam. The adjustment mechanism is coupled to the beam and is configured to enable adjustment of the beam along at least one translational or angular direction. The probe is directed toward a cavity entrance of the body lumen using the adjustment mechanism. The central probe unit is maintained sealingly coupled to the cavity entrance by propelling the distal carriage along the beam. The evacuation adapter is maintained sealingly coupled to the probe by propelling the central carriage along the beam, concurrent with the displacement of the hollow tube relative to the central probe unit. Lumen material is evacuated from the body lumen using the evacuation adaptor. The tubular medical instrument is advanced and retracted within the body lumen by propelling the proximal carriage along the beam. The system may further include a control unit, configured to control the distal drive, the central drive, and the proximal drive. The adjustment mechanism may include: height adjuster, configured to adjust the height of the beam; a yaw angle adjuster, configured to adjust a rotational yaw angle of the beam; a pitch angle adjuster, configured to adjust a rotational pitch angle of the beam; and/or a roll angle adjuster, configured to adjust a rotational roll angle of the beam. The system may further include sensors disposed on the central carriage, and configured to detect a ring disposed on the hollow tube, where the displacement of the translation mechanism carriages is synchronized based on the detection to position the hollow tube in relation to the evacuation adapter. The system may further include force measuring sensors, configured to measure: forces induced between at least one of the translation mechanism carriages, and/or forces affecting the body lumen by at least one of: the tubular medical instrument and the probe, where the movement of at least one of the translation mechanism carriages is restricted in accordance with the measured forces to remain within a selected range. At least one of: the endoscope adapter; and the swivel fastener unit, may include a fastener, configured selectively to firmly grip the shaft or to release the shaft to enable free movement thereof. The fastener may be inflatable and deflatable via a port, where inflation of the fastener enables firmly gripping the shaft, and where deflation of the fastener enables releasing of the shaft. The tubular medical instrument may include: an angulation controller, configured to enable controlling an angular positioning of a tip of the tubular medical instrument, and/or a rotation controller, configured to enable controlling a rotation of the shaft of the tubular medical instrument. The tubular medical instrument may be an endoscope. The hollow tube may include a sleeve-covered-sheath, and the central probe unit may include a central valve unit operational for inflating and deflating the sleeve-covered-sheath.

In accordance with another aspect of the present invention, there is thus provided a method for advancing and retracting a tubular medical instrument within a body lumen of a patient and evacuating lumen material therefrom. The method includes the procedures of: mounting a probe onto a distal carriage of a translation mechanism, the probe including a hollow tube and a central probe unit through which the hollow tube is moveably inserted; mounting an evacuation adaptor at a proximal end of the probe onto a central carriage of the translation mechanism, and mounting a swivel fastener unit at a proximal end of the evacuation adapter onto a proximal carriage of the translation mechanism. The method further includes the procedure of inserting a shaft of the tubular medical instrument through a central bore of the probe, the evacuation adapter, and the swivel fastener unit. The method further includes the procedure of directing the probe toward a cavity entrance of the body lumen, using an adjustment mechanism coupled to a beam of the translation mechanism and configured to enable adjustment of the beam along at least one translational or angular direction. The method further includes the procedures of maintaining the central probe unit sealingly coupled to the cavity entrance by propelling the distal carriage along the beam, and maintaining the evacuation adapter sealingly coupled to the probe by propelling the central carriage along the beam, concurrent with the displacement of the hollow tube relative to the central probe unit. The method further includes the procedures of evacuating lumen material from the body lumen using the evacuation adaptor, and advancing and retracting the tubular medical instrument within the body lumen by propelling the proximal carriage along the beam. A control unit may be used to control the distal drive, the central drive, and the proximal drive. The displacement of at least one of: the distal carriage; the central carriage; and the proximal carriage, may be synchronized to position the hollow tube in relation to the evacuation adapter, based on a detection of a ring disposed on the hollow tube, using sensors disposed on the central carriage. The movement of at least one of: the distal carriage; the central carriage; and the proximal carriage, may be restricted in accordance with measured forces, to maintain the measured forces within a selected range, the measured forces including: forces induced between at least one of the translation mechanism carriages, and/or forces affecting the body lumen by at least one of: the tubular medical instrument and the probe. At least one of: the endoscope adapter; and the swivel fastener unit, may include a fastener, configured selectively to firmly grip the shaft or to release the shaft to enable free movement thereof. The fastener may be inflatable and deflatable via a port, where inflation of the fastener enables firmly gripping the shaft, and where deflation of the fastener enables releasing of the shaft. An angular positioning of a tip of the tubular medical instrument may be controlled using an angulation controller of the tubular medical instrument, and a rotation of the shaft of the tubular medical instrument may be controlled using a rotation controller of the tubular medical instrument. The tubular medical instrument may be an endoscope. The hollow tube may include a sleeve-covered-sheath, and the central probe unit may include a central valve unit operational for inflating and deflating the sleeve-covered-sheath.

### BRIEF DESCRIPTION OF THE DRAWINGS

The present invention will be understood and appreciated more fully from the following detailed description taken in conjunction with the drawings in which:

FIG. 1 is an illustration of a system for advancing and retracting a tubular medical instrument within a body lumen of a patient and evacuating lumen material, constructed and operative in accordance with an embodiment of the present invention;

FIG. 2 is a perspective view illustration of the system of FIG. 1 mounted on a treatment table and positioned for treating a patient, constructed and operative in accordance with an embodiment of the present invention;

FIG. 3 is an enlarged view illustration of a portion of the system of FIG. 1, constructed and operative in accordance with an embodiment of the present invention;

FIG. 4 is an enlarged view illustration of the swivel fastener unit of the system of FIG. 1, embracing an endoscope shaft, constructed and operative in accordance with an embodiment of the present invention;

FIG. 5A is a perspective view illustration of the swivel fastener unit of FIG. 4, constructed and operative in accordance with an embodiment of the present invention;

FIG. 5B is a perspective exploded view illustration of the swivel fastener unit of FIG. 4, constructed and operative in accordance with an embodiment of the present invention;

FIG. 5C is a perspective view illustration of a cut out swivel plug of the swivel fastener unit of FIG. 4, constructed and operative in accordance with an embodiment of the present invention;

FIG. 6 is an enlarged view illustration of the probe, the evacuation adaptor, and the swivel fastener unit of the system of FIG. 1, constructed and operative in accordance with an embodiment of the present invention;

FIG. 7 is an enlarged view illustration of the evacuation adaptor of the system of FIG. 1, constructed and operative in accordance with an embodiment of the present invention;

FIG. 8A is an enlarged cross-sectional view illustration of a portion of the evacuation adaptor of FIG. 7, constructed and operative in accordance with an embodiment of the present invention;

FIG. 8B is an enlarged cross-sectional view illustration of another portion of the evacuation adaptor of FIG. 7, constructed and operative in accordance with an embodiment of the present invention;

FIG. 9 is an enlarged view illustration of the probe central valve unit of the system of FIG. 1, constructed and operative in accordance with an embodiment of the present invention;

FIG. 10A is a perspective stand-alone view illustration of a conventional endoscope arranged for operation in conjunction with components of the system of FIG. 1, constructed and operative in accordance with an embodiment of the present invention;

FIG. 10B is a perspective view illustration of a conventional endoscope inserted through the swivel plug of the system of FIG. 1, constructed and operative in accordance with an embodiment of the present invention;

FIG. 10C is a perspective view illustration of a conventional endoscope inserted through the swivel plug, the evacuation adaptor tube, and the probe central valve unit, of the system of FIG. 1, constructed and operative in accordance with an embodiment of the present invention;

FIG. 11 is a schematic illustration of the control unit of the system of FIG. 1, constructed and operative in accordance with an embodiment of the present invention; and

FIG. 12 is a block diagram of a method for advancing and retracting a tubular medical instrument within a body lumen of a patient and evacuating lumen material, operative in accordance with another embodiment of the present invention.

### DETAILED DESCRIPTION OF THE EMBODIMENTS

The present invention overcomes the disadvantages of the prior art by providing a system and method for advancing and retracting a tubular medical instrument, such as an endoscope, within a body cavity of a patient, for purposes of examination, diagnosis, and/or treatment of the patient. The disclosed system and method are intended to facilitate the insertion and extraction of the endoscope within a body lumen, in a manner that reduces the efforts required by an operator and enhances the effectiveness of the examination, diagnosis and/or treatment procedure.

The term “tubular medical instrument” is used herein to refer to any type of medical device or apparatus configured for insertion into a body lumen or internal cavity, in the context of a medical procedure, such as for examination, diagnosis or treatment purposes. Examples of a tubular medical instrument may include, but are not limited to: an endoscope; a colonoscope; a cystoscope; a bronchoscope; a catheter; and a guidewire. The tubular medical instrument may include supplementary devices or accessories for assisting with the operation of the medical procedure, such as: an imaging device, a light source, a power source, and the like.

The terms “user” and “operator” are used interchangeably herein to refer to any individual person or group of persons using or operating the system of the present invention, such as a nurse, a physician or other medical practitioner certified to perform an endoscopic procedure (i.e., an endoscopist).

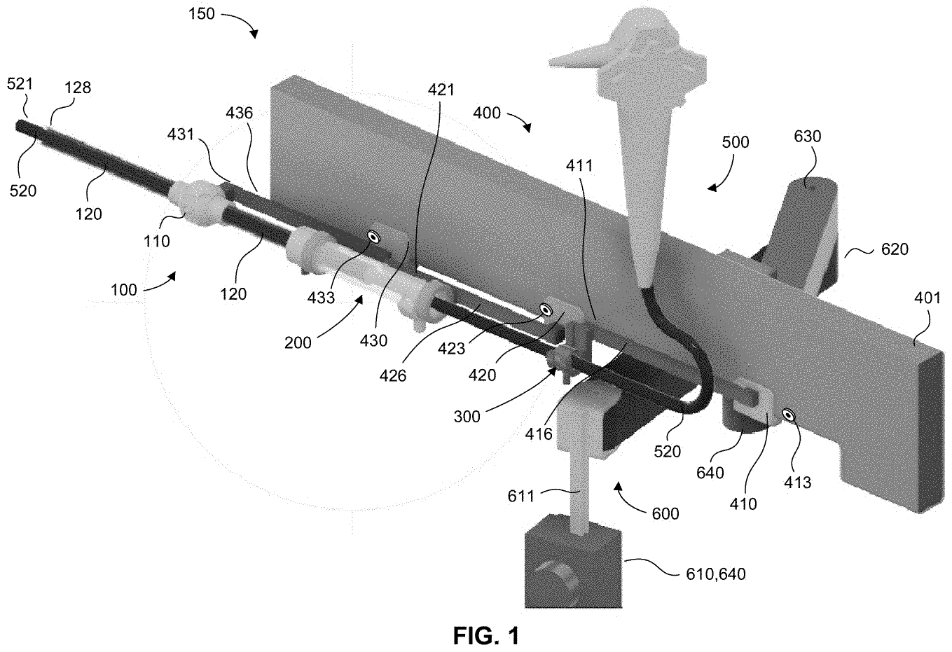

Reference is now made to FIG. 1, which is a schematic illustration of a system, generally referenced 150, for advancing and retracting a tubular medical instrument within a body lumen of a patient and evacuating lumen material. System 150 is depicted and described herein for exemplary purposes in the context of propelling an endoscope, but may alternatively be configured to advance and retract a different type of tubular medical instrument. Similarly, the body lumen is depicted and described herein for exemplary purposes as being the colon (large intestine) and/or additional passageways in the gastrointestinal tract, but may alternatively represent other body cavities, such as the throat or lungs. System 150 may also be utilized to advance or retract instruments in lumens and other regions for non-medical applications as well.

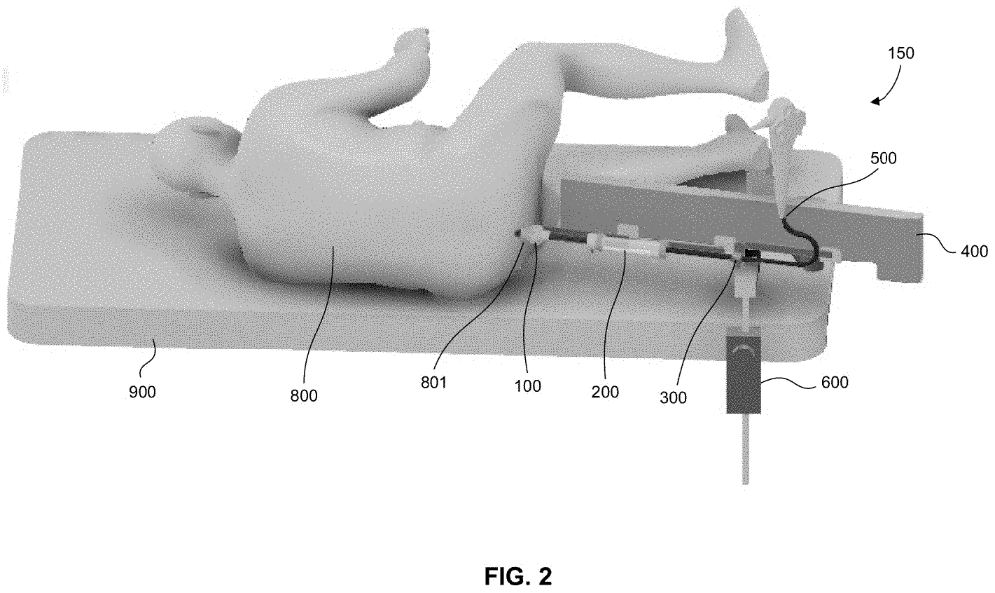

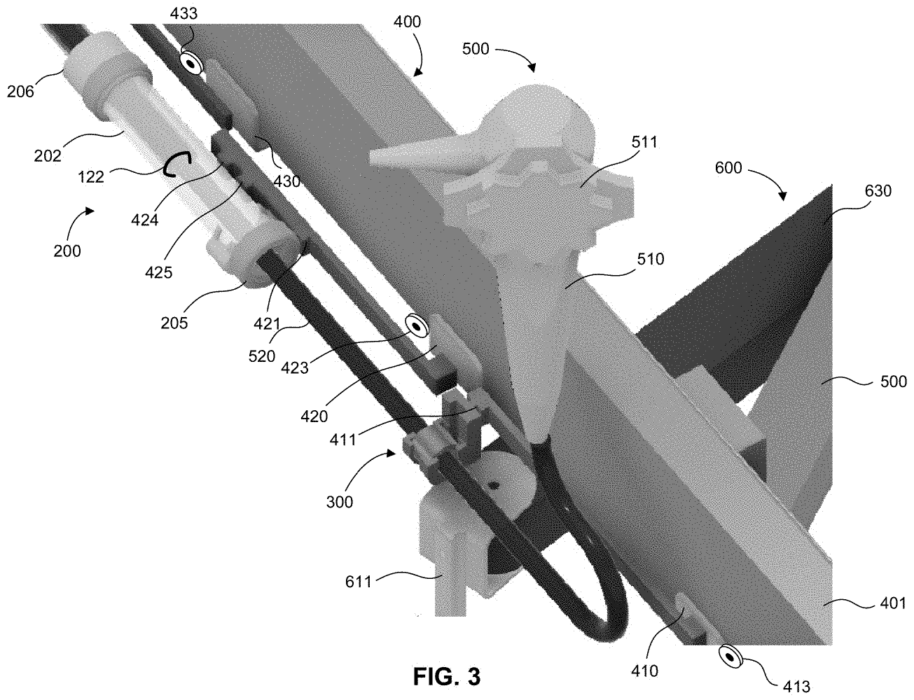

System 150 includes a probe 100, an evacuation adapter 200, a swivel fastener unit 300, a linear translation mechanism 400, an endoscope 500, an adjustment mechanism 600, and a control unit 700. Evacuation adapter 200 is coupled with probe 100 and with swivel fastener unit 300. Linear translation mechanism 400 is mounted on adjustment mechanism 600. Control unit 700 is communicatively coupled with evacuation adapter 200, and linear translation mechanism 400. Adjustment mechanism 600 is generally mounted on or adjacent to a treatment surface, such as a bed or table, on which the patient is positioned when performing the medical procedure. Reference is further made to FIGS. 2 and 3. FIG. 2 is a perspective view illustration of system 150 mounted on a treatment table, referenced 900, and positioned for treating a patient, referenced 800. FIG. 3 is an enlarged view illustration of a portion of system 150.

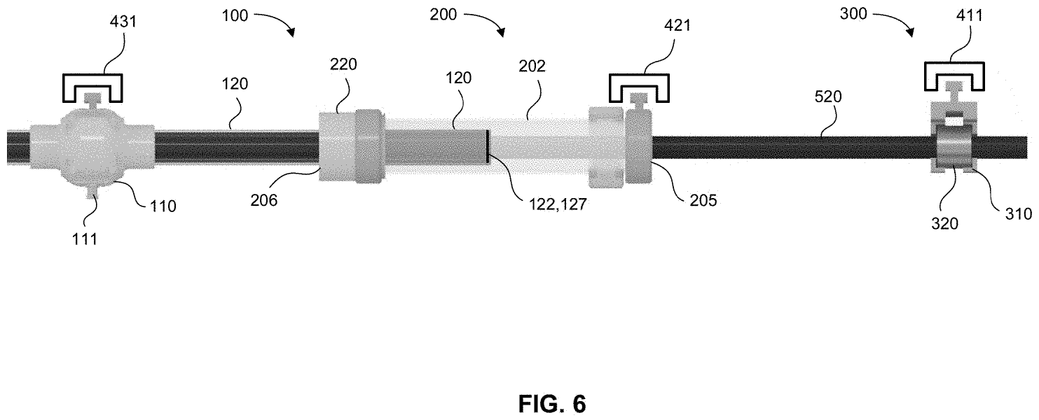

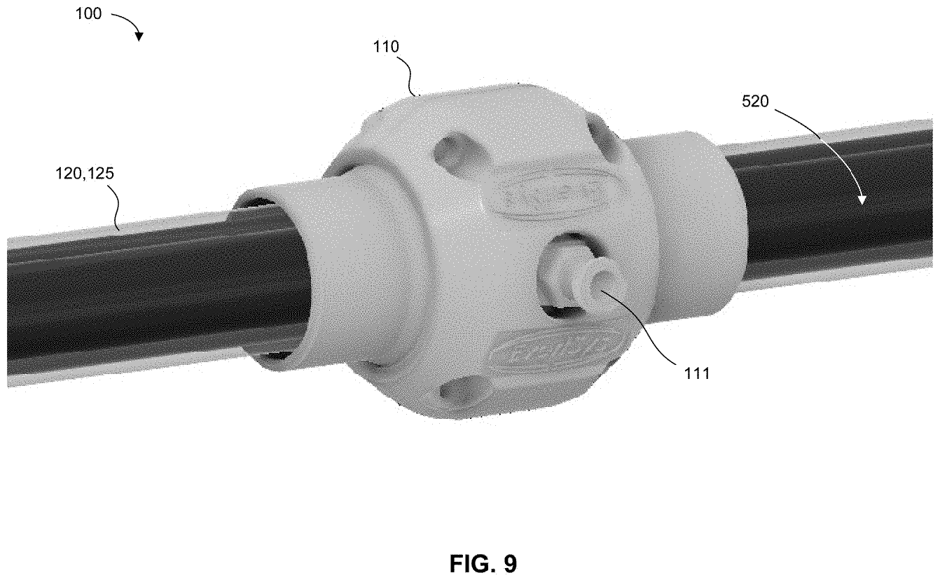

Probe 100 relates to a hollow component which is advanceable through a body lumen by inflating a flexible sleeve, and is operational to conduct at least part of an endoscope through the probe, such as the probe disclosed in WO2016/075682A1 to Lichtenstein, entitled “Hollow probe with sleeve”. Probe 100 includes a central valve unit 110, a fluid control port 111, a tubular sheath 121, and a flexible sleeve 125. Reference is also made to FIG. 6, which is an enlarged view illustration of the probe (100), the evacuation adaptor (200), and the swivel fastener unit (300) of the system (150) of FIG. 1. Tubular sheath 121 is slidably displaceable (i.e., in a proximal or distal direction) within a central bore of central valve unit 110. Tubular sheath 121 is covered by flexible sleeve 125 to form a sleeve-covered-sheath, referenced 120. In particular, flexible sleeve 125 is inserted within tubular sheath 121 and inverted over the sheath edges to fully envelop sheath 121, covering the distal and proximal ends thereof and forming sleeve-covered-sheath 120, which defines a sleeve-covered-sheath proximal side 127 and a sleeve-covered-sheath distal side 128. Sleeve-covered-sheath 120 represents an example of a hollow tube of probe 100, which may alternatively be embodied by a different type of hollow tube (e.g., without a sleeve or a sheath) The central bore of tubular sheath 121 is open to accommodates a portion of a medical instrument, such as endoscopic shaft 520 of endoscope 500, which is maintained isolated from the internal face of sheath 121 by sleeve 125. Accessories or supplementary components for facilitating the operation of endoscope 500 may also be positioned within the central bore of tubular sheath 121, such as wires, or cables, which may be coupled to endoscopic shaft 520 and may extend to the operator or to equipment outside of patient. Sleeve 125 is also sealingly fastened to the distal and proximal ends of central valve unit 110. Sleeve 125 may be composed of a flexible material, such as biocompatible plastic, and impermeable to fluids that may be introduced into sleeve 125 through central valve unit 110. Sleeve 125 together with central valve unit 110 sealingly envelop sheath 121, which is completely enshrouded by sleeve 125 and valve unit 110, and contains the introduced fluid while allowing slidable displacement of sheath 121. A metal ring 122 is disposed on the proximal side 127 of sleeve-covered-sheath 120. Sleeve 125 may be inflated or deflated via a fluid control port 111 of central valve unit 110, such as using a pressurized gas or liquid, to propel probe 110 forward. Central valve unit 110 represents an example of a central probe unit of probe 100, which may be an alternative type of central unit (e.g., without a valve). Simultaneously or alternatively, sheath 121 may be propelled manually on its proximal end via a manual or mechanical propulsion. The inflation of sleeve 125 may be controlled so that sleeve 125 is gradually fed out of sheath 121. A portion of sleeve 125 that is inflated may expand radially outward and remain substantially stationary relative to the body lumen (e.g., intestinal wall) while the sleeve remains inflated, without rubbing the internal walls of the lumen, avoiding frictional damage or trauma to the lumen and enabling substantially frictionless advancement and retraction of the probe within the lumen. Reference is further made to FIG. 9, which is an enlarged view illustration of the probe central valve unit (110) of the system (150) of FIG. 1.

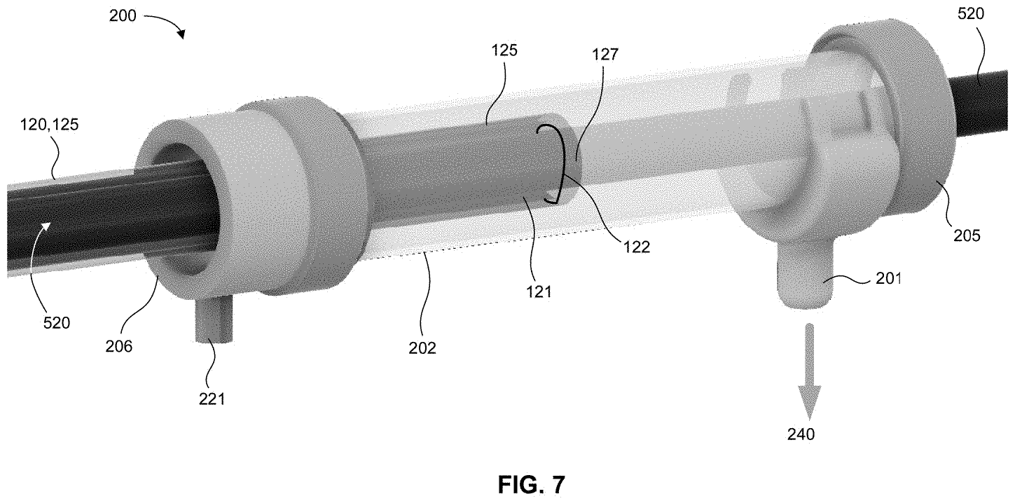

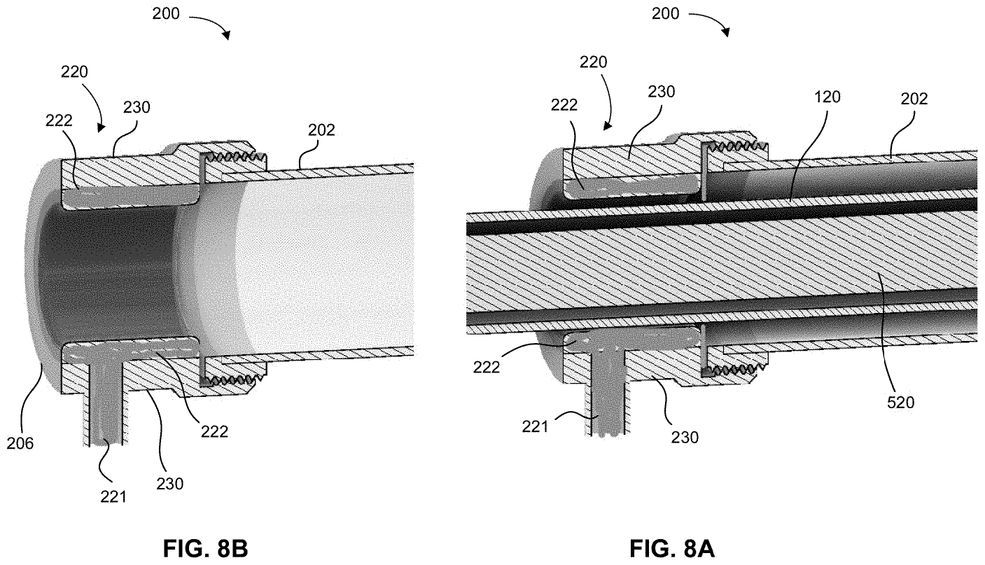

Referring back to FIG. 1, evacuation adapter 200 is a component operational to allow evacuation of body fluids or lumen material from a body lumen while allowing insertion of at least part of an endoscope through the adapter and probe, such as the evacuation adapter disclosed in WO2019/077609A1 to Lichtenstein, entitled “Hollow probe with sleeve”. Endoscope adapter 200 is sealinlgy mounted to proximal side 127 of sleeve-covered-sheath 120 (e.g., using a sealing ring not shown), allowing insertion of endoscope 500 through endoscope adapter 200 and through sleeve-covered-sheath 120, while sealing gaps between adapter 200 and endoscope 500 (on the proximal side) and between adapter and sleeve-covered-sheath 120 (on the distal side) to retain the evacuated lumen material and prevent their leakage through gaps. Evacuation adapter 200 includes an evacuation adapter suction port 201, an evacuation adapter tube 202, and an evacuation adapter fastener 220. Evacuation adapter tube 202 defines an evacuation adapter tube proximal side 205 and an evacuation adapter tube distal side 206. Reference is further made to FIGS. 6, 7, 8A and 8B. FIG. 7 is an enlarged view illustration of the evacuation adaptor (200) of the system (150) of FIG. 1. FIG. 8A is an enlarged cross-sectional view illustration of a portion of evacuation adaptor 200, and FIG. 8B is an enlarged cross-sectional view illustration of another portion of evacuation adaptor 200.

Evacuation adapter suction port 201 is in fluid communication with a suction mechanism 240, and is operational for the removal of fluid material, such as liquid or fecal particles in the gastrointestinal tract, by a suction process, where the fluid material is disposed in the cavity confined between evacuation adaptor tube 202, endoscope 500, and sleeve-covered-sheath 120. Evacuation adapter fastener 220 includes a rigid cylinder 230 and an internal inflatable cushion in the form of a toroidal balloon 222 (best seen in FIGS. 8A and 8B). Evacuation adapter fastener toroidal balloon 222 may be inflated to firmly grip endoscope shaft 520, or deflated to release endoscope shaft 520 for allowing the displacement of endoscope shaft 520 therethrough. The inflation and deflation of evacuation adapter fastener toroidal balloon 222 is conducted via a fluid (e.g., air) conduit entering rigid cylinder 230 of evacuation adapter fastener 220 at an evacuation adapter fastener control port 221, where the inflow and outflow of fluid through evacuation adapter fastener control port 221 is controlled by control unit 700.

Referring back to FIG. 1, linear translation mechanism 400 includes a beam 401 with three carriages movable along the beam: a proximal carriage 410, a central carriage 420 and a distal carriage 430. Each carriage 410, 420, 430 is equipped with a respective fast release connection 411, 421, 431. Probe 100, evacuation adapter 200, and swivel fastener unit 300 are each mounted on respective translation mechanism carriages 410, 420, 430. In particular, central valve unit 110 of probe 100 is mounted onto distal carriage 430 (depicted as leftmost carriage in the Figures). Evacuation adapter 200 is mounted onto central carriage 420 by means of mounting arm 426. Swivel cradle 310 of swivel fastener unit 300 is mounted onto proximal carriage 410 (depicted as rightmost carriage in the Figures) by means of mounting arm 416.

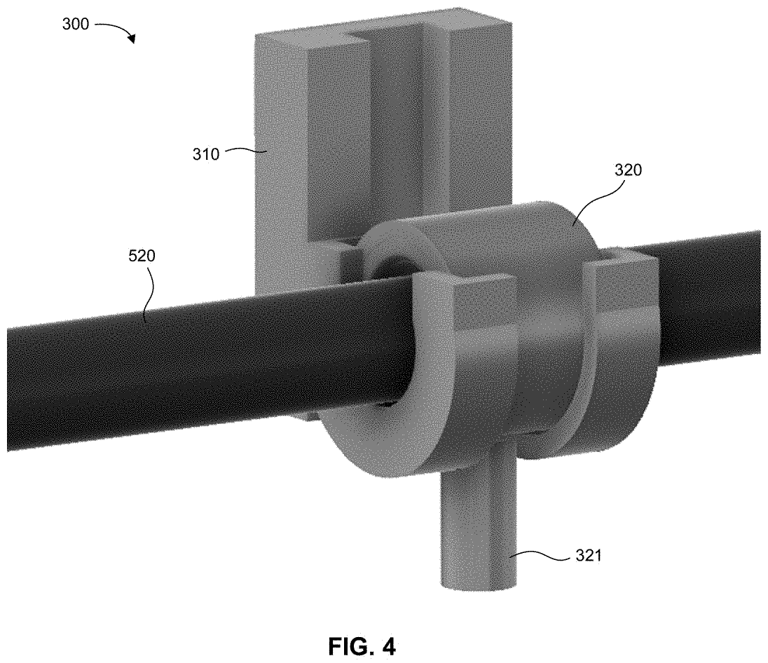

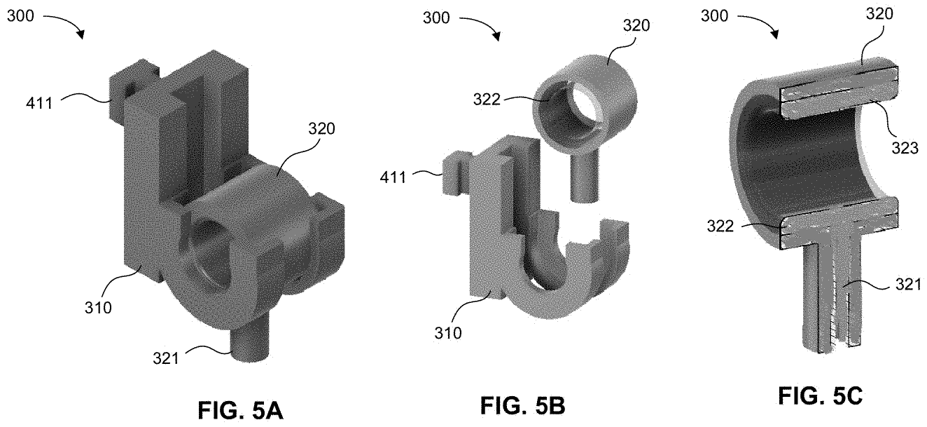

Endoscope shaft 520 passes through a tubular bore of swivel plug 320 of swivel fastener unit 300. Swivel plug 320 includes a swivel plug control port 312 and a swivel plug fastener 322. Reference is further made to FIGS. 4, 5A and 5B. FIG. 4 is an enlarged view illustration of the swivel fastener unit (300) of the system (150) of FIG. 1. FIG. 5A is a perspective view illustration of swivel fastener 300 unit; FIG. 5B is a perspective exploded view illustration of swivel fastener unit 300, and FIG. 5C is a perspective view illustration of a cut out swivel plug 320 of swivel fastener unit 300.

Swivel plug fastener 322 is configured to enable the advancement or retraction of endoscope shaft 520 (i.e., distally or proximally), while enabling the operator to rotate endoscope shaft 520 (i.e., about its longitudinal axis) using endoscope handle 510. The release of swivel plug fastener 322 enables proximal carriage 410 to slide over endoscope shaft 520, without moving endoscope shaft 520. Swivel plug 320 is nested in a swivel cradle 310, which is mounted on proximal carriage 410. Swivel plug 320 is formed as a rigid cylinder encircling swivel plug fastener 322. Swivel plug fastener 322 includes an internal inflatable cushion in the form of a toroidal balloon 323, which may be inflated to firmly grip endoscope shaft 520, or deflated to release endoscope shaft 520 for allowing the displacement of endoscope shaft 520 therethrough. The inflation and deflation of swivel plug fastener toroidal balloon 323 is conducted via a fluid (e.g., air) conduit entering swivel plug 320 at swivel plug control port 321, where the inflow and outflow of fluid through swivel plug control port 321 is controlled by control unit 700.

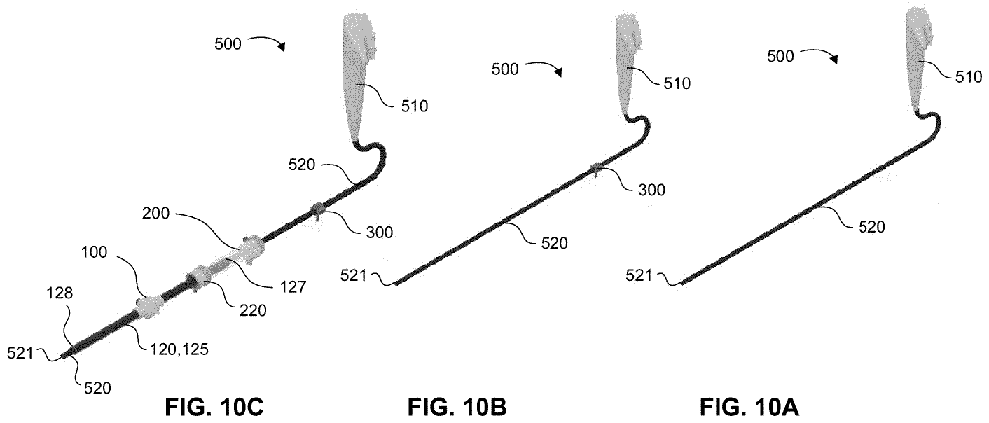

Endoscope 500 is a conventional endoscope, as known in the art. Endoscope 500 includes an endoscope handle 510 with endoscope angulation knobs 511, and an endoscopic shaft 520 defined by an endoscope tip 521 at the distal end thereof. Endoscope handle 510 allows the operator to rotate endoscopic shaft 520 (i.e., about its longitudinal axis). Endoscope angulation knobs 511 allows the operator to control the angulation (i.e., angular positioning) of endoscope tip 521. Endoscope 500 may also include supplementary devices or accessories (not shown) for assisting with the endoscopic procedure, such as: an imaging device, a light source, a power source, and the like. Reference is made to FIGS. 10A, 10B and 10C. FIG. 10A is a perspective stand-alone view illustration of a conventional endoscope 500 arranged for operation in conjunction with components of system 150. FIG. 10B is a perspective view illustration of a conventional endoscope 500 inserted through the swivel plug 320 of system 150. FIG. 10C is a perspective view illustration of a conventional endoscope 500 inserted through the swivel plug 320, the evacuation adaptor tube 202, and the probe central valve unit 110, of system 150.

Linear translation mechanism 400 is mounted on adjustment mechanism 600. Adjustment mechanism 600 is configured to enable an adjustment of the position and orientation of probe 100, endoscope adapter 200 and/or endoscope 500 via adjustment of beam 401 of linear translation mechanism 400. In particular, adjustment mechanism 600 includes a height adjuster 610, a yaw adjuster 620, a pitch adjuster 630, and a roll adjuster 640. Height adjuster 610 includes a height control arm 611. Yaw adjuster 620 is coupled to height control arm 611 along a first axis. Pitch adjuster 620 is coupled to yaw adjuster 620 along a second axis. Roll adjuster 640 is coupled to pitch adjuster 630 along a third axis. Beam 401 is mounted onto roll adjuster 640. Accordingly, height adjuster 610 is configured to adjust the height (i.e., move the position of beam 401 up or down) via movement of height control arm 611. Yaw adjuster 620 is configured to adjust the rotational yaw angle (e.g., about the first coupling axis) of beam 401, pitch adjuster 630 is configured to adjust the rotational pitch angle (e.g., about the second coupling axis) of beam 401, while roll adjuster 640 is configured to adjust the rotational roll angle (e.g., about the third coupling axis) of beam 401. It is noted that height, pitch, yaw and roll adjustments are exemplary, and may alternatively be substituted with equivalent translational or rotational directional adjustments along six degrees of freedom (e.g., inclined yaw, lateral translation in a horizontal or a slanted plane, and the like). The adjustment of the height and orientation of beam 401 using adjustment mechanism 600 allows the operator to position probe in relation to the body lumen cavity entrance 801 of patient 800 during the initial stages of the endoscopic procedure. Adjustment mechanism 600 may be considered to enable a crude adjustment of the endoscopic components (e.g., probe 100, endoscope adapter 200, swivel fastener unit 300, endoscope 500), while linear translation mechanism 400 may be considered to enable a finer adjustment. Adjusting mechanism 600 may be mounted on treatment table 900 (FIG. 2), or on an adjacent external surface, such as a cart.

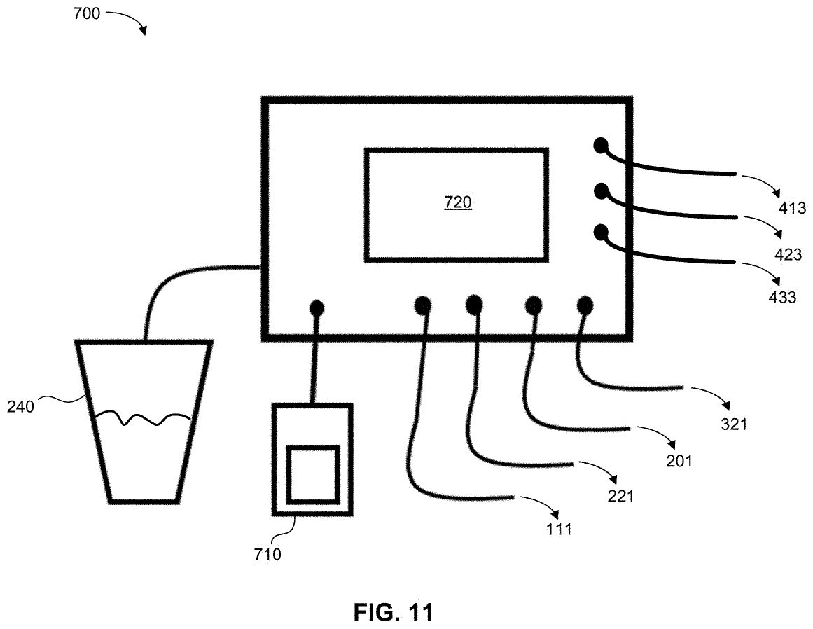

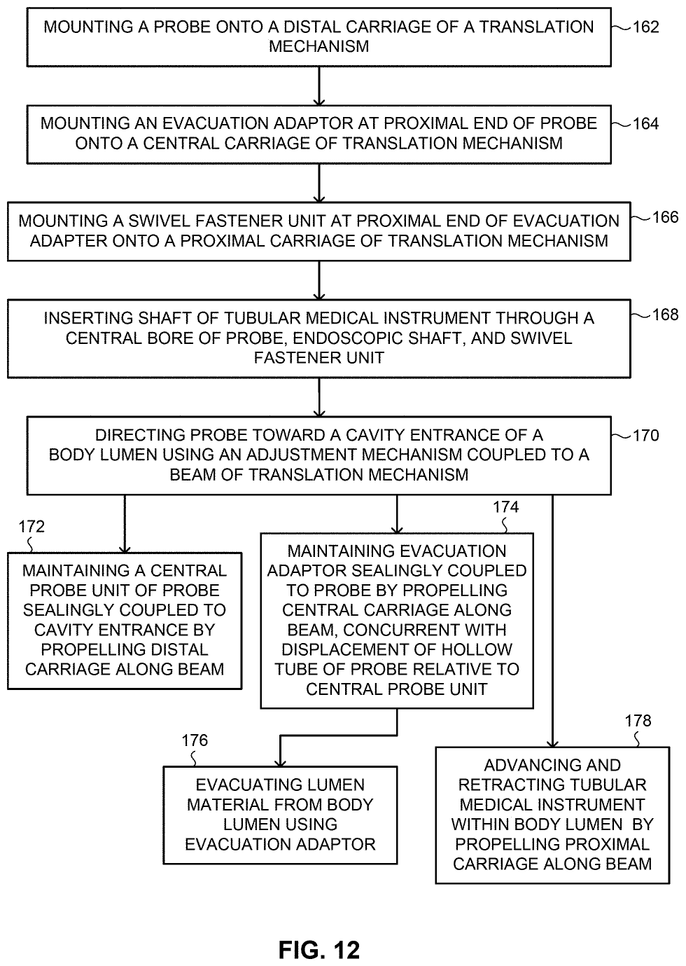

Reference is made to FIG. 11, which is a schematic illustration of control unit 700 of system 150. Control unit 700 includes a foot pedal 710 and a touchscreen 720. It is appreciated that foot pedal 710 represents an exemplary input interface and may alternatively be substituted with a different type of tactile interface (e.g., a hand-based interface). Touchscreen 700 is a display-based interface that allows the operator to enter commands via manual gestures as well as to receive visual and/or audio information (e.g., text, images, symbols) on a display screen, but may similarly be substituted with one or more alternative input/output interfaces. Control unit 700 allows the operator to activate and terminate the movement of translation mechanism 400 in general, and translation mechanism carriages 410, 420, 430 in particular. Each translation mechanism carriage 410, 420, 430 features a respective propelling drive (e.g., wheels, rollers, an endless track, a magnetic rail, and the like) configured to propel the carriage (i.e., along a proximal or distal direction). Proximal carriage 410 includes a proximal carriage drive 413; central carriage 420 includes a central carriage drive 423; and distal carriage 430 includes a distal carriage drive 433 (depicted in FIG. 3). Each carriage drive 413, 423, 433 is independently controlled by control unit 700 (under the direction of the operator using foot pedal 710 or touchscreen 720), to activate or terminate the movement of a respective translation mechanism carriage 410, 420, 430. The movement of translation mechanism carriages 410, 420, 430 is synchronized, such that, for example, the distal advancement of endoscopic shaft 520 via proximal carriage 410 is linked to a corresponding distal advancement of center carriage, so as to ensure that sleeve-covered-sheath proximal side 127 will be positioned within evacuation adapter tube 202, approximately in the center of evacuation adapter 200. The synchronization is enabled by virtue of using sensors 424, 425 disposed on central carriage 420 near the center of evacuation adapter tube 202. Sensors 424, 425 are configured to detect metal ring 122 disposed on sleeve-covered-sheath proximal side 127 (i.e., on the proximal end of sheath 121).

Each translation mechanism carriage 410, 420, 430 may include additional force measuring sensors (not shown), configured to measure the forces induced between translation mechanism carriage 410, 420, 430, and the forces affecting the body lumen walls by endoscope tip 521 and/or by sleeve-covered-sheath distal side 128. The measured forces may be utilized by control unit 700, such as by allowing or restricting the movement of certain translation mechanism carriages 410, 420, 430, via their respective carriage propelling drives 413, 423, 433, such that the measured forces remain confined within allowed or desirable ranges. The measured forces and the relative movement of the translation mechanism carriages 410, 420, 430 may also be indicated to the operator (e.g., via touchscreen 720), who may control each carriage propelling drive 413, 423, 433 accordingly.

System 150 may optionally include and/or be associated with additional components not shown in the Figures for enabling the implementation of the disclosed subject matter.

The operation of system 150 will now be described in general terms, followed by specific examples. System 150 is initialized such that translation mechanism carriages 410, 420, 430 are brought to their default positions (in relation to the other system components). The operator positions evacuation adapter 200 on probe 100, and mounts central valve unit 110 of probe 100 onto distal carriage 430 (using fast release connection 431), and mounts evacuation adapter 200 onto central carriage 420 via mounting arm 426 (using fast release connection 421). The operator positions swivel plug 320 in swivel cradle 310, and mounts swivel cradle 310 onto proximal carriage 410 via mounting arm 416 (using fast release connection 411). The operator then inserts endoscopic shaft 520 into the central bore or tubular opening of swivel cradle 310, swivel plug 320, evacuation adapter tube 202, and sleeve-covered-sheath 120 of probe 100. Subsequently, the operator directs endoscopic tip 521 toward body lumen cavity entrance 801 of patient 800, using height adjuster 610, yaw adjuster 620, pitch adjuster 630, and roll adjuster 640 of adjustment mechanism 600.

At this stage, the operator can advance endoscopic tip 521 into body lumen cavity entrance 801 using foot pedal 710 or touchscreen 730 of control unit 700 to activate the movement of respective translation mechanism carriages 410, 420, 430, while controlling the angulation (i.e., angular positioning) of endoscope tip 521 using endoscope angulation knobs 511, and the rotation of endoscopic shaft 520 using endoscope handle 510. The distal advancement of proximal carriage 410 causes a corresponding distal advancement of endoscopic shaft 520, and a corresponding partial advancement of probe 100 in the distal direction. The actual advancement of probe 111 is dependent on the friction between endoscopic shaft 520 and sleeve 125 as well as the inflation status of sleeve 125 (i.e., whether sleeve 125 is deflated or inflated). The uncertainty in the exact positioning of probe 100 may be rectified using sensors 424 and 425 on center carriage 420. Control unit 700 activates center carriage 420 to advance according to the readings of sensors 424 and 425 so as to ensure that sleeve-covered-sheath proximal side 127 will be disposed approximately in the center of evacuation adapter 200.

Analogously, in order to withdraw or retract endoscopic shaft 520 from the body lumen, the operator directs proximal carriage 410 to move in a proximal direction (using foot pedal 710 or touchscreen 730 of control unit 700), causing a corresponding proximal retraction of probe 100. Control unit 700 activates the retraction of center carriage 420 according to the readings of sensors 424 and 425 so as to ensure that sleeve-covered-sheath proximal side 127 will be disposed approximately in the center of evacuation adapter 200.

In order to advance or retract endoscopic shaft 520 without moving probe 100, sleeve 125 is inflated or deflated via control port 111, and evacuation adapter fastener toroidal balloon 222 is inflated via control port 221, to stabilize sleeve-covered-sheath 120 of probe 100 within evacuation adaptor tube 202. Similarly, swivel plug fastener toroidal balloon 323 may be inflated via control port 321, to firmly grip endoscopic shaft 520 by swivel plug fastener 322. Thereafter, the operator can advance or retract endoscopic shaft 520 by moving proximal carriage 410 (using foot pedal 710 or touchscreen 730 of control unit 700), and using endoscope angulation knobs 511 to angulate endoscopic tip 521 and using endoscope handle 510 to rotate endoscopic shaft 520. The operator can retract probe 100 and endoscope 500 together, while inspecting the gastrointestinal tract of patient 800, and moving the translation carriages 410, 420, 430 in a proximal direction. The operator may deploy evacuation adaptor 200 to remove fluid material, such as fecal particles in the gastrointestinal tract, through evacuation adapter suction port 201, by activating suction mechanism 240 using control unit 700.

The operator may remove endoscope 500 from the body lumen at any stage of the endoscopic procedure by retracting probe 100 by inflating/deflating sleeve 125 via control port 111. Evacuation adapter fastener toroidal balloon 222 can be deflated via control port 221, to release endoscopic shaft 520 from evacuation adapter fastener 220 and allow its proximal displacement therethrough. Similarly, swivel plug fastener toroidal balloon 323 can be deflated via control port 321, to release endoscopic shaft 520 from swivel plug fastener 322 and allow its proximal displacement therethrough. The operator may detach or remove endoscope 500 from the other system components (probe 100, evacuation adapter 200, swivel fastener unit 300) while moving translation mechanism 400 and adjustment mechanism 600 away from the working area, and continue to perform the endoscopic procedure manually.

Reference is now made to FIG. 5, which is a block diagram of a method for advancing and retracting a tubular medical instrument within a body lumen of a patient and evacuating lumen material, operative in accordance with an embodiment of the present invention. In procedure 162, a probe is mounted onto a distal carriage of a translation mechanism. Referring to FIGS. 1 and 3, the operator positions evacuation adapter 200 on probe 100, and mounts central valve unit 110 of probe 100 onto distal carriage 430 of translation mechanism 400 using fast release connection 431.

In procedure 164, an evacuation adaptor at a proximal end of the probe is mounted onto a central carriage of the translation mechanism. Referring to FIGS. 1 and 3, the operator mounts evacuation adapter 200 onto central carriage 420 of translation mechanism 400 via mounting arm 426 using fast release connection 421.

In procedure 166, a swivel plug fastener at a proximal end of the evacuation adapter is mounted onto a proximal carriage of the translation mechanism. Referring to FIGS. 1 and 3, the operator positions swivel plug 320 in swivel cradle 310 of swivel fastener unit 300, and mounts swivel cradle 310 onto proximal carriage 410 of translation mechanism 400 via mounting arm 416 using fast release connection 411.

In procedure 168, a shaft of a tubular medical instrument is inserted through a central bore of the probe, the evacuation adapter, and the swivel fastener unit. Referring to FIGS. 1 and 3, the operator inserts endoscopic shaft 520 of endoscope 500 into the central bore or tubular opening of swivel cradle 310 and swivel plug 320 of swivel fastener unit 300, through the evacuation adapter tube 202 of evacuation adapter 200, and through sleeve-covered-sheath 120 (or hollow tube) of probe 100.

In procedure 170, the probe is directed toward a cavity entrance of a body lumen using an adjustment mechanism coupled to a beam of the translation mechanism. Referring to FIGS. 1, 2 and 3, the operator directs endoscopic tip 521 toward body lumen cavity entrance 801 of patient 800, using height adjuster 610, yaw adjuster 620, pitch adjuster 630, and roll adjuster 640 of adjustment mechanism 600 to adjust the height and rotational angles of beam 401 of translation mechanism 400, where beam 401 is mounted onto at least a portion of adjustment mechanism 600.

In procedure 172, a central probe unit of the probe is maintained sealingly coupled to the cavity entrance by propelling the distal carriage along the beam. Referring to FIGS. 1, 2 and 3, the operator advances endoscopic tip 521 into the body lumen of patient 800 by activating carriage propelling drives 413, 423, 433 to propel the displacement of respective translation mechanism carriages 410, 420, 430, using control unit 700. Probe 100 is advanced distally by activating distal carriage drive 431 to propel distal carriage 430. The distal advancement of distal carriage 430 is controlled to maintain central valve unit 110 (or other central probe unit) of probe 100 sealingly coupled to body lumen cavity entrance 801 of patient 800.

In procedure 174, the evacuation adaptor is maintained sealingly coupled to the probe by propelling the central carriage along the beam concurrent with the displacement of a hollow tube of the probe relative to the central probe unit. Referring to FIGS. 1, 2 and 3, the operator advances endoscopic tip 521 into the body lumen of patient 800 by activating carriage propelling drives 413, 423, 433 to propel the displacement of respective translation mechanism carriages 410, 420, 430, using control unit 700. Evacuation adapter 200 is advanced distally by activating central carriage drive 421 to propel central carriage 420. The distal advancement of central carriage 420 is controlled to maintain sleeve-covered sheath 120 (or other hollow tube of probe 100) sealingly coupled to evacuation adaptor tube 202. Control unit 700 may activate center carriage 420 to advance according to the readings of sensors 424 and 425 so as to ensure that sleeve-covered-sheath proximal side 127 will be disposed approximately in the center of evacuation adapter 200.

In procedure 176, lumen material is evacuated from within the body lumen using the evacuation adaptor. Referring to FIGS. 2, 3 and 7, the operator activates evacuation adaptor 200 to remove fluid material from the body lumen of patient 800, such as to remove fecal particles from the gastrointestinal tract of patient 800, through evacuation adapter suction port 201, by activating suction mechanism 240 using control unit 700.

In procedure 178, the shaft of the tubular medical instrument is advanced and retracted by propelling the proximal carriage along the beam of the translation mechanism. Referring to FIGS. 1, 2 and 3, the operator advances endoscopic tip 521 into the body lumen of patient 800 by activating carriage propelling drives 413, 423, 433 to propel the displacement of respective translation mechanism carriages 410, 420, 430, using control unit 700. Swivel fastener unit 300 holding a proximal end of endoscopic shaft 500 is advanced distally by activating proximal carriage drive 411 to propel proximal carriage 410. The operator can control the angular positioning of endoscope tip 521 using endoscope angulation knobs 511, and can control the rotation of endoscopic shaft 520 using endoscope handle 510. Sleeve 125 of probe 100 may be inflated or deflated via control port 111 in order to advance or retract endoscopic shaft 520 without moving probe 100. Evacuation adapter fastener toroidal balloon 222 may be inflated via control port 221 so as to firmly grip endoscopic shaft 520 by evacuation adapter fastener 220, or deflated so as to release endoscopic shaft 520 from evacuation adapter fastener 220 and allow displacement of endoscopic shaft 520 relative to evacuation adapter 200. Swivel plug fastener toroidal balloon 323 may be inflated via control port 321 so as to firmly grip endoscopic shaft 520 by swivel plug fastener 322, or deflated so as to release endoscopic shaft 520 from swivel plug fastener 322 and allow displacement of endoscopic shaft 520 relative to swivel fastener unit 300.

While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. A system for advancing and retracting a tubular medical instrument within a body lumen and evacuating lumen material therefrom, the system comprising:
a probe, comprising a hollow tube and a central probe unit through which the hollow tube is moveably inserted, the probe configured to hold a shaft of the tubular medical instrument;
an evacuation adaptor, disposed proximal to the probe, and configured to hold the shaft of the tubular medical instrument, and to enable the evacuation of lumen material;
a swivel fastener unit, disposed proximal to the probe, and configured to hold the shaft of the tubular medical instrument;
a translation mechanism, comprising:
a beam; and a plurality of translation mechanism carriages comprising:
a distal carriage, configured to mount the probe, the distal carriage comprising a distal drive configured to propel the displacement of the distal carriage along the beam;
a central carriage, configured to mount the evacuation adaptor, the central carriage comprising a central drive configured to propel the displacement of the central carriage along the beam; and
a proximal carriage, configured to mount the swivel fastener unit, the proximal carriage comprising a proximal drive configured to propel the displacement of the proximal carriage along the beam; and
an adjustment mechanism, coupled to the beam and configured to enable adjustment of the beam along at least one translational or angular direction,
wherein the probe is directed toward a cavity entrance of the body lumen using the adjustment mechanism, the central probeunit is maintained sealingly coupled to the cavity entrance by propelling the distal carriage along the beam, the evacuation adapter is maintained sealingly coupled to the probe by propelling the central carriage along the beam, concurrent with the displacement of the hollow tube relative to the central probe unit, wherein lumen material is evacuated from the body lumen using the evacuation adaptor, and wherein the tubular medical instrument is advanced and retracted within the body lumen by propelling the proximal carriage along the beam.

2. The system of claim 1, further comprising a control unit, configured to control the distal drive, the central drive, and the proximal drive.

3. The system of claim 1, wherein the adjustment mechanism comprises at least one of:
height adjuster, configured to adjust the height of the beam;
a yaw angle adjuster, configured to adjust a rotational yaw angle of the beam;
a pitch angle adjuster, configured to adjust a rotational pitch angle of the beam; and
a roll angle adjuster, configured to adjust a rotational roll angle of the beam.

4. The system of claim 1, further comprising sensors disposed on the central carriage, the sensors configured to detect a ring disposed on the hollow tube, wherein the displacement of the translation mechanism carriages is synchronized based on the detection to position the hollow tube in relation to the evacuation adapter.

5. The system of claim 1, further comprising force measuring sensors, configured to measuring at least one force selected from the group consisting of:
forces induced between at least one of the translation mechanism carriages; and
forces affecting the body lumen by at least one of: the tubular medical instrument and the probe,
wherein the movement of at least one of the translation mechanism carriages is restricted in accordance with the measured forces to remain within a selected range.

6. The system of claim 1, wherein at least one of: the endoscope adapter; and
the swivel fastener unit, comprises a fastener, configured selectively to firmly grip the shaft or to release the shaft to enable free movement thereof.

7. The system of claim 6, wherein the fastener is inflatable and deflatable via a port, wherein inflation of the fastener enables firmly gripping the shaft, and wherein deflation of the fastener enables releasing of the shaft.

8. The system of claim 1, wherein the tubular medical instrument comprises at least one of:
an angulation controller, configured to enable controlling an angular positioning of a tip of the tubular medical instrument; and
a rotation controller, configured to enable controlling a rotation of the shaft of the tubular medical instrument.

9. The system of claim 1, wherein the tubular medical instrument is an endoscope.

10. The system of claim 1, wherein the hollow tube comprises a sleeve-covered-sheath, and wherein the central probe unit comprises a central valve unit operational for inflating and deflating the sleeve-covered-sheath.

11. A method for advancing and retracting a tubular medical instrument within a body lumen and evacuating lumen material therefrom, the method comprising the procedures of:
mounting a probe onto a distal carriage of a translation mechanism, the probe comprising a hollow tube and a central probe unit through which the hollow tube is moveably inserted;
mounting an evacuation adaptor at a proximal end of the probe onto a central carriage of the translation mechanism;
mounting a swivel fastener unit at a proximal end of the evacuation adapter onto a proximal carriage of the translation mechanism;
inserting a shaft of the tubular medical instrument through a central bore of the probe, the evacuation adapter, and the swivel fastener unit;
directing the probe toward a cavity entrance of the body lumen, using an adjustment mechanism coupled to a beam of the translation mechanism and configured to enable adjustment of the beam along at least one translational or angular direction;
maintaining the central probe unit sealingly coupled to the cavity entrance by propelling the distal carriage along the beam;
maintaining the evacuation adapter sealingly coupled to the probe by propelling the central carriage along the beam, concurrent with the displacement of the hollow tube relative to the central probe unit;
evacuating lumen material from the body lumen using the evacuation adaptor; and
advancing and retracting the tubular medical instrument within the body lumen by propelling the proximal carriage along the beam.

12. The method of claim 11, further comprising operating a control unit to control at least one of:
a distal drive configured to propel the displacement of the distal carriage along the beam;
a central drive configured to propel the displacement of the central carriage along the beam; and
a proximal drive configured to propel the displacement of the proximal carriage along the beam.

13. The method of claim 11, wherein the displacement at least one of: the distal carriage; the central carriage; and the proximal carriage, is synchronized to position the hollow tube in relation to the evacuation adapter, based on a detection of a ring disposed on the hollow tube, using sensors disposed on the central carriage.

14. The method of claim 11, wherein the movement of at least one of: the distal carriage; the central carriage; and the proximal carriage, is restricted in accordance with measured forces, to maintain the measured forces within a selected range, the measured forces selected from the group consisting of:
forces induced between at least one of the translation mechanism carriages; and
forces affecting the body lumen by at least one of: the tubular medical instrument and the probe.

15. The method of claim 11, wherein at least one of: the endoscope adapter;
and the swivel fastener unit, comprises a fastener, configured selectively to firmly grip the shaft or to release the shaft to enable free movement thereof.

16. The method of claim 15, wherein the fastener is inflatable and deflatable via a port, wherein inflation of the fastener enables firmly gripping the shaft, and wherein deflation of the fastener enables releasing of the shaft.

17. The method of claim 11, further comprising at least one procedure selected from the group consisting of:
controlling an angular positioning of a tip of the tubular medical instrument, using an angulation controller of the tubular medical instrument; and
controlling a rotation of the shaft of the tubular medical instrument, using a rotation controller of the tubular medical instrument.

18. The method of claim 11, wherein the tubular medical instrument is an endoscope.

19. The method of claim 11, wherein the hollow tube comprises a sleeve-covered-sheath, and wherein the central probe unit comprises a central valve unit operational for inflating and deflating the sleeve-covered-sheath.
